(19) United States
(12) Patent Application Publication
(10) Pub. No.: US 2026/0191591 A1
(43) Pub. Date: Jul. 9, 2026

(54) REAL-TIME SURGICAL SUPPORT METHOD USING SENSOR-BASED AI SEGMENTATION MODEL AND COMPUTER SYSTEM AND RECORDING MEDIUM FOR PERFORMING THE SAME

(71) Applicant: Kyung-Woo Park, Seoul (KR)

(72) Inventor: Kyung-Woo PARK, Seoul (KR)

(21) Appl. No.: 19/074,463

(22) Filed: Mar. 10, 2025

(30) Foreign Application Priority Data
Jan. 8, 2025 (KR) 10-2025-0002614

(51) Int. Cl. A61B 34/10; A61B 17/34; G06T 7/10

(52) U.S. Cl. CPC A61B 34/10; A61B 17/3403; G06T 7/10; A61B 2034/101; A61B 2034/107; G06T 2207/10116; G06T 2207/30012

(57) ABSTRACT

A real-time surgery support computer system may include a scout image collection unit that collects an AP and lateral images of an anatomical structure of a spine by using C-arm equipment in order to perform supervised learning, an AI segmentation model unit that generates a 2-D image by learning the AP and lateral images and then generates a lateral image from an AP image, a sensor data and AP/lateral image synchronization module that synchronizes AP/lateral images of the AI segmentation model unit and data of position and pressure sensors mounted on surgical instrument, an instrument moving tracking module that visualizes the data of the position and pressure sensors synchronized with the AP/lateral images by tracking the data of the position and pressure sensors, and an instrument moving prediction simulation module that predicts and simulates a movement of the surgical instrument moving in the instrument moving tracking module in real time.

### CROSS-REFERENCE TO RELATED APPLICATION

This application claims priority under 35 U.S.C. § 119 to Korean Patent Application No. 10-2025-0002614 filed on Jan. 8, 2025, which is incorporated herein by reference in its entirety.

### TECHNICAL FIELD

The present disclosure relates to a surgical support method capable of analyzing a spinal-related disease and increasing the accuracy of a surgical procedure by using an artificial intelligence (AI) segmentation model and a computer system for performing the surgical support method, and more particularly, to a real-time surgical support method using a sensor-based AI segmentation model, which can recognize an anatomical structure by segmenting the anatomical structure from an anteroposterior (AP) image and lateral image of C-arm equipment, generate a lateral image (or AP image) and the position of surgical instrument from the AP image (or the lateral image) by using a deep learning model and a three-dimensional (3-D) transformation model, and support a process of performing a spinal disease surgery by monitoring a process of tracking information (e.g., an incident angle, a moving distance, and a direction) related to a movement of the surgical instrument, and a computer system and a recording medium for performing the real-time surgical support method.

### BACKGROUND ART

Today, in advanced countries including Korea, various diseases are changed into chronic disease structures since the advanced countries enter an aging society. In particular, in the aging society, a spinal disease and a heart disease appear as troublesome chronic diseases. A spine has a structure in which several segments from cervical vertebrae to thoracic vertebrae, lumbar vertebrae, sacral vertebrae, and coccygeal vertebra are complex, and is a passage through which nerves that control all over the body pass. Accordingly, spinal diseases are present with a wide range of symptoms and regions. As a result, prompt diagnosis and timely treatment are required, but a surgical procedure process is complicated and an advanced surgical technique is required like a spine structure. Accordingly, a burden of a patient and a surgical operator is increased. In order to reduce such a burden of a spinal disease patient and a surgical procedure of a surgical operator, research regarding a precise diagnosis and surgical procedure, the prevention and treatment of a disease through the prediction of prognosis, and the management of the entire cycle through the integration and analysis of personal medical records and various medical data, are actively performed around a medical convergence center.

In spinal disease research, many image data necessary for radiological diagnosis and treatment are used. Both a patient and a surgical operator are in danger and increasingly avoid a radiological surgery because equipment itself for securing images use radiation and thus the patient and the surgical operator are inevitably naturally exposed to radiation.

In order to perform percutaneous lumbar extra-foraminotomy (hereinafter referred to as “PLEF”) for reducing a pain that is rooted in a lumbar vertebrae part, among spinal diseases, an image of the C-arm equipment is essential. Accordingly, a high degree of a surgical procedure technique is necessary to perform a surgery while minimizing X-ray image photographing.

The PLEF is one of surgical procedures for securing a nerve passage that has been narrowed by peeling off a ligament entangled in intervertebral foramen and then removing a cause of a waist or leg pain that occurs due to the compression of nerves attributable to foraminal stenosis or spinal stenosis by injecting a pain-relieving medication.

Intervertebral foramen through clinical dissection includes spine nerves (dorsal root ganglion (DRG)), sinuvertebral nerves, interverbral veins, radicular veins, an artery, and ligament flavum.

FIG. 1 is a distribution diagram of ligaments around intervertebral foramen. As illustrated in FIG. 1, ligaments around the intervertebral foramen are classified into the ligaments of an entrance zone, the ligaments of a mid-zone, the ligaments of an exit zone, and the ligaments of a posterior zone of a tube. The ligaments of the entrance zone consist of a posterior longitudinal ligament, a Hoffmann ligament, and a peridural membrane. The ligaments of the mid-zone consist of fascial condensations that attach a nerve root sleeve to pedicles and ligamentum flavum. The ligaments of the exit zone (around intervertebral foramen) consist of an internal ligament, a transforaminal ligament, and an external ligament. The ligaments of the posterior zone of the tube consist of cribriform fascia.

In a PLEF process, a surgical procedure is performed by avoiding a dangerous area while alternately keeping a close eye on an AP image and a lateral image photographed through C-arm equipment. Accordingly, the surgical process is very complicated, and a high degree of concentration is required. In particular, in the existing PLEF, the accuracy of a surgery is greatly different according to a surgeon because the surgery is performed depending on a surgeon's experience and senses.

For example, in a process of performing a PLEF surgery, an incident angle or position moving of a surgical instrument depend on a surgeon's experience and feeling. However, a burden of both a patient and a surgical operator are inevitably increased because surgical accuracy is very different depending on a surgeon's skill.

As a scheme for reducing liability for risk of such PLEF and improving the accuracy of a surgery, attempts to graft AI were made. However, a PLEF surgery method using AI has not been embodied because it is very difficult to generate an image data set.

Prior Technology 1 is disclosed in Korean Patent No. 2140393 (entitled “METHOD OF ANALYZING LUMBAR SPINE IN RADIOGRAPHIES BASED ON ARTIFICIAL INTELLIGENCE, RECORDING MEDIUM AND DEVICE FOR PERFORMING THE METHOD”). As illustrated in FIG. 2, Prior Technology 1 is a technology capable of automating an image analysis task by identifying and labelling a lumbar vertebrae region from many images by using a deep learning neural network algorithm.

However, the technology is limited to separating and analyzing a lumbar vertebrae region from many images secured in a diagnosis process, that is, a step prior to a surgical procedure process, and thus does not substantially help much on the side that the position of instrument needs to be immediately adjusted while watching an X-ray image that is photographed in real time in a surgical procedure process.

As another Prior Technology 2, U.S.A. Patent Application Publication No. 2021-0290315 (entitled “SYSTEM METHOD FOR COMPUTER-ASSISTANT SURGERY AND COMPUTER PROGRAM PRODUCT THEREFOR”) is disclosed. As illustrated in FIG. 3, Prior Technology 2 provides a light projector 300 configured to project at least one pattern onto a spine and includes a three-dimensional (3-D) video camera 201 configured to capture a 3-D video image of a spine and pattern when the spine comes into sight. In Prior Technology 2, the display of an output tool state for a current direction and position of a tool 202 that is used in a spine surgery is repeatedly computerized through an inertial navigation system (INS). Furthermore, data communication between a sub-system and a processor is provided by transmitting the display of the output tool state to the processor through a tool tracker 201 including a wireless communication module. The processor receives the display of the output tool state generated by the tool tracker and a 3-D video image, and tracks a spine based on a pattern that has been known to the processor, thereby providing feedback to a surgeon. Prior Technology 2 embodies an advantage of approaching to target position correctly by tracking the surgical tool 200 that is moved by being inserted into the spine.

In addition, as Prior Technology 3, Korean Patent Application Publication No. 2021-0127849 (entitled “SYSTEM FOR DETERMINING SEVERITY OF ANKYLOSING SPONDYLITIS USING ARTIFICIAL INTELLIGENCE BASED ON SPINAL IMAGE AND METHOD THEREOF”) is disclosed. As Prior Technology 4, Korean Patent No. 2553060 (entitled “METHOD, APPARATUS AND PROGRAM FOR PROVIDING MEDICAL IMAGE USING SPINE INFORMATION BASED ON AI”) is disclosed. As Prior Technology 5, Korean Patent Application Publication No. 2023-0137581 (entitled “SYSTEM AND METHOD FOR AUTOMATIC MEASUREMENT OF SPINAL PARAMETERS BASED ON ARTIFICIAL INTELLIGENCE POINT DETECTION”) is disclosed. These prior technologies are focused on obtaining information on which the state or prognosis of a spine disease can be determined by extracting spine-related information by reading a photographed image and grafting the spine-related information onto AI.

### RELATED ART DOCUMENT

### Patent Document

Korean Patent No. 2140393 (Jul. 27, 2020)
U.S.A. Patent Application Publication No. US 2021/0290315A1 (Sep. 23, 2021)
Korean Patent Application Publication No. 2021-0127849 (Oct. 25, 2021)
Korean Patent No. 2553060 (Jul. 4, 2023)
Korean Patent Application Publication No. 10-2023-0137581 (Oct. 5, 2023)

### SUMMARY OF THE INVENTION

Various embodiments are directed to provide a real-time surgical support method using a sensor-based artificial intelligence (AI) segmentation model, which can generate an accurate lateral segmentation image (or anteroposterior (AP) segmentation image) from an AP image (or lateral image) of C-arm equipment by using a learning algorithm in which a three-dimensional (3-D) transformation mathematical formula and a classification/dimension reduction model in a minimally invasive surgery technique, such as PLEF, and a computer system for performing the real-time surgical support method.

Furthermore, various embodiments are directed to provide a real-time surgical support method using a sensor-based AI segmentation model, which can track and monitor a movement of surgical instrument in real time by synchronizing AP/lateral segmentation images of the C-arm equipment, which are generated by accurately segmenting an anatomical structure, and signals detected by the position sensor and pressure sensor of surgical instrument by using a 3-D transformation mathematical formula and a classification/dimension reduction learning model, and a computer system for performing the real-time surgical support method.

Furthermore, various embodiments are directed to provide a real-time surgical support method using a sensor-based AI segmentation model, which can actually implement a process of tracking a movement of surgical instrument in AP/lateral segmentation images in a minimally invasive PLEF process, and a computer system for performing the real-time surgical support method.

Furthermore, various embodiments are directed to provide a real-time surgical support method using a sensor-based AI segmentation model, which can support a process of performing a surgery by a surgeon by using AP/lateral images as a fixing screen based on an AI segmentation model and learning a process of tracking a movement of the position of surgical instrument in the fixing screen in real time, and a computer system for performing the real-time surgical support method.

In an embodiment, a real-time surgery support computer system using a sensor-based artificial intelligence (AI) segmentation model includes a scout image collection unit configured to collect an anteroposterior (AP) image and lateral image of an anatomical structure of a spine by using C-arm equipment in order to perform supervised learning, an AI segmentation model unit configured to generate a two-dimensional (2-D) image by learning the AP and lateral images collected by the scout image collection unit and then generate an lateral image from an AP image, a sensor data and AP/lateral image synchronization module configured to synchronize AP/lateral images generated through the AI segmentation model unit and data of a position sensor and pressure sensor mounted on surgical instrument, an instrument moving tracking module configured to visualize the data of the position sensor and pressure sensor synchronized with the AP/lateral images by tracking the data of the position sensor and pressure sensor, and an instrument moving prediction simulation module configured to predict and simulate a movement of the surgical instrument that moves in the instrument moving tracking module in real time.

In an embodiment of the present disclosure, the real-time surgery support computer system further includes a data record and analysis module configured to store and analyze data generated by the instrument moving prediction simulation module in order to use the data in learning so that a moving path of the surgical instrument is able to be predicted more accurately after a surgery.

In an embodiment of the present disclosure, the AI segmentation model unit includes a deep learning module configured to generate the 2-D image and a three-dimensional (3-D) image generation module configured to generate the lateral image from the AP image by applying a mathematical formula model, a 3-D transformation model, and a classification/minimum dimension model.

In an embodiment of the present disclosure, the deep learning module combines and trains a CNN model and an LSTM model.

In an embodiment of the present disclosure, the surgical instrument includes a trocar including a needle including a groove formed in a predetermined part and a long and thin pole having a pointed needle tip at a front end thereof, a handle into which the needle is inserted and configured to apply a force so that directionality, straightness, and steerability are facilitated based on a percutaneous incident angle of a spinal facet joint, a position sensor installed on one side of the handle and configured to generate coordinate data, and a pressure sensor installed on the other side of the handle and configured to generate a force pattern.

In an embodiment of the present disclosure, the real-time surgery support computer system further includes a mapping module configured to receive the coordinate data and data of the force pattern generated by the position sensor and pressure sensor of the trocar and to map the coordinate data and data of the force pattern to the AP and lateral images. In this case, the mapping module includes a computer main body configured to match the AP and lateral images and to perform a computing task, a monitor configured to display AP and lateral images mapped in the computer main body, and a keyboard configured to perform an operation of stopping a position of the surgical instrument in order to designate coordinate data when the surgical instrument with which the AP and lateral images of the AI segmentation model unit are overlaid is moved.

Furthermore, in a second embodiment of the trocar, the surgical instrument includes a needle including a groove formed in a predetermined part and a long and thin pole having a pointed needle tip at a front end thereof, a handle into which the needle is inserted and configured to apply a force so that directionality, straightness, and steerability are facilitated based on a percutaneous incident angle of a spinal facet joint, a position sensor installed on one side of the handle and configured to generate coordinate data, and a pressure sensor installed on the other side of the handle and configured to generate a force pattern.

In this case, the handle includes a cylinder including two space parts, an adaptor coupled to one side of the cylinder and into which the needle is inserted, and a hammer anvil installed on a cross section of the cylinder and configured to apply hammering in intervertebral foramen.

In a second embodiment of the handle, the pressure sensor is installed in the space part of the cylinder on one side thereof. The space part of the cylinder on the other side thereof is formed as a pressure transfer space. A shock buffer is installed in the pressure transfer space and generates a signal by reducing strength of striking pressure of the hammering that is transferred to the pressure sensor.

In this case, a sensor connector is mounted on the space part of the cylinder on the other side thereof. A circuit board and a battery are embedded in the sensor connector. Furthermore, a cover is installed at an end of the cylinder on one side thereof. A position sensor, a power switch, LED lamps for indicating a charging state, and an antenna for communicating with the AI segmentation model unit are mounted on the cover.

In a second embodiment of the handle of the present disclosure, a predetermined interval is formed between the adaptor and the pressure transfer space so that pressure is collected in the pressure transfer space and transferred to the needle when the striking for the hammering is applied to the cylinder in the state in which the adaptor and the pressure sensor have not come into direct contact with each other.

The position sensor may include a load cell and a gyroscope in order to generate position coordinates.

Furthermore, the position sensor and the pressure sensor that are installed in the handle are connected to a mapping module and transmit a position signal and a pressure signal.

In this case, the mapping module includes a computer main body configured to match the AP and lateral images and to perform a computing task, a monitor configured to display AP and lateral images mapped in the computer main body, and a keyboard configured to perform an on/off operation for designating a position of the surgical instrument in order to designate reference coordinate data when the surgical instrument with which the AP and lateral images of the AI segmentation model unit are overlaid is moved.

The AI segmentation model unit according to an embodiment of the present disclosure includes one or more learning model selected, among a transformer model, 3-D convolutional neural networks (3-D CNNs), a recurrent convolutional neural network (RCNN), generative adversarial networks (GANs), attention mechanisms, autoencoders, and reinforced learning.

In an embodiment of the present disclosure, a real-time surgical support method using a sensor-based artificial intelligence (AI) segmentation model includes a first step of collecting scout anteroposterior (AP) and lateral images of a spine from C-arm equipment, a second step of classifying and labelling each tissue region of an anatomical structure by pre-processing the scout AP and lateral images, a third step of obtaining AP and lateral images by learning the labelled scout AP and lateral images in an AI segmentation model, a fourth step of obtaining position data of instrument from a scout AP image that is initially collected, mapping the position data of the instrument to a scout lateral image, and visualizing the position data of the instrument, a fifth step of designating a reference point of the position data of the instrument in the scout lateral image, a sixth step of proposing an optimal moving path of the instrument to a surgical operator through a chatbot function of the AI segmentation model after performing the fifth step, a seventh step of determining whether the instrument has deviated its moving path after performing the sixth step, generating warning when the instrument deviates a safe region, and automatically correcting the moving path, if necessary, an eighth step of obtaining, by an instrument movement simulation module, force pattern data from a pressure sensor while continuously simulating the moving path of the instrument until a trocar reaches a target position, after performing the seventh step, and a ninth step of determining, by a surgical operator upon monitoring, whether the images of the C-arm equipment are matched with the scout images by overlaying the images of the C-arm equipment, performing corrections until the images of the C-arm equipment are matched with the scout images when the images of the C-arm equipment are not matched with the scout images, and simulating a movement of the instrument when the images of the C-arm equipment are matched with the scout images.

In an embodiment of the present disclosure, the learning of the AI segmentation model includes a first process of learning the AP/lateral images of the anatomical structure through a CNN algorithm and an LSTM algorithm that are included in a deep learning module, a second process of learning an AP image and lateral image generated by a three-dimensional (3-D) image generation module and generating a lateral image (or an AP image) from the AP image (or the lateral image) by using a mathematical formula algorithm and a 3-D transformation algorithm, a third process of integrating the AP image and the lateral image so that a surgical operator is able to view the AP image and the lateral image on one screen, and a fourth process of completing a scout segmentation image by integrating the AP image and the lateral image.

In an embodiment of the present disclosure, the designating of the reference point of the position data comprises designating a hammer point, a facet line, and a swing position which are classification areas of the anatomical structure.

Furthermore, in an embodiment of the present disclosure, the pressure sensor detects pressure that is applied to a specific tissue, and provides feedback for excessive pressure to the surgical operator in real time when the excessive pressure occurs.

Furthermore, in an embodiment of the present disclosure, in order to accurately track a movement of the instrument based on data of a position sensor and the pressure sensor, the AI segmentation model understands and learns a process by labelling a movement of the instrument in a coded language.

Furthermore, in an embodiment of the present disclosure, when a movement of the trocar deviates in an unexpected direction or pressure of the trocar is suddenly changed, the AI segmentation model immediately detects the movement or the pressure and indicates warning in order to prevent an insertion of the trocar in a wrong direction or excessive pressure of the trocar.

In an embodiment of the present disclosure, the real-time surgical support method further includes a tenth step of storing and analyzing data of a position sensor and the pressure sensor generated in a surgical process and information collected from an AI segmentation model and the AP and lateral images in order to store and analyze data of predicting a moving path of the instrument.

In an embodiment of the present disclosure, there is provided a computer-readable recording medium on which a computer program for performing the real-time surgical support method using a sensor-based AI segmentation model has been recorded.

As described above, according to embodiments of the present disclosure, the following effects are embodied.

First, a lateral segmentation image (or an AP image) indicative of an anatomical structure of a spine can be generated through learning to which the 3-D transformation mathematical formula and the classification/dimension reduction algorithm have been applied with respect to segmentation image data labelled by inputting a scout image necessary for supervised learning to the base model and segmenting a region of an AP image (or lateral image) of the C-arm equipment for the spine.

Second, AP/lateral segmentation images can be displayed by generating AP/lateral segmentation images (or lateral/AP segmentation images) based on only the AP image (or lateral image) of the C-arm equipment and generating a signal detected by the position sensor of surgical instrument as 3-D coordinate values (i.e., an incident angle and moving length) of the surgical instrument.

Third, a labelling image having a learnable form can be generated with respect to results by using a matching module in which AP/lateral segmentation images of the C-arm equipment and position and pressure data of surgical instrument have been synchronized. Accordingly, a surgery time can be significantly reduced and surgical accuracy can be improved because a movement of the surgical instrument in the AP/lateral segmentation images can be tracked in real time.

Fourth, according to an embodiment of the present disclosure, an exposure dose that is exposed by radioactive photographing can be significantly reduced because AP/lateral segmentation images can be displayed at the same time based on only one AP image (or lateral image) of the C-arm equipment.

Fifth, a real-time movement of surgical instrument can be tracked in AP/lateral segmentation images of the C-arm equipment, and the accuracy and safety of surgery can be improved because such a tracking process can be monitored.

Sixth, it is possible to apply an embodiment of the present disclosure to various surgical operations that require a 3-D image (e.g., transforming a plan (or side) image into a side (or plan) image). That is, an embodiment of the present disclosure is not limited to a spine surgery like spinal fusion and may be expanded to medical fields, such as an artificial joint replacement surgery, traumatic therapy, and a cardiac stent insertion technology.

Seventh, according to an embodiment of the present disclosure, the diversity and accuracy of AI learning can be maximized by sharing the data with worldwide medical institutions through the cloud server because learnable labelling image data can be generated by integrating an image of the C-arm equipment, position sensor and pressure sensor data, and an image of an image changeover using the AI segmentation model and the tracking of a movement of surgical instrument can be monitored in the learnt image. In particular, the system according to an embodiment of the present disclosure can improve the performance because the system is self-trained consistently by being connected to the cloud server.

Eighth, an image of the C-arm equipment and the AI segmentation model can be integrated, and labelling learning using the data of the position sensor and pressure sensor of surgical instrument is possible. Accordingly, a picture of a digital camera having a flexible structure in which additional data can be coupled can be easily combined.

Ninth, the precision and applicability of an AI learning model can be expanded by integrating sensor data with a picture of a multi-angled digital camera in addition to an image of the C-arm equipment.

Tenth, real-time bi-directional feedback in a surgical process is possible using the AI segmentation model. That is, the AI segmentation model and a surgical operator can commune with each other in real time, a movement of surgical instrument continues to be corrected, and labelling is performed. Accordingly, the accuracy of data can be improved, and data generated through feedback can contribute to constructing a more precise learning model.

### BRIEF DESCRIPTION OF THE DRAWINGS

FIG. 1 is a distribution diagram of ligaments around intervertebral foramen.

FIG. 2 is a concept view illustrating a method of analyzing a lumbar vertebrae region using AI according to a conventional technology.

FIG. 3 is a construction diagram of a computer-assistant surgery system capable of tracking a tool by using a navigation system according to a conventional technology.

FIG. 4 is a schematic diagram illustrating a computer system for supporting a real-time surgery using a sensor-based AI segmentation model according to an embodiment of the present disclosure.

FIG. 5 is a block diagram illustrating components of the computer system for supporting a real-time surgery using the sensor-based AI segmentation model according to an embodiment of the present disclosure.

FIG. 6 is a concept view of a first deep learning process of the AI segmentation model, that is, a main part of the present disclosure.

FIG. 7 is a concept view of a second deep learning process of the AI segmentation model, that is, a main part of the present disclosure.

FIG. 8 is a comparison diagram of an actual AP image that is generated through the first and second deep learning processes and a prediction image, with respect to an AP image of a spine photographed in C-arm equipment.

FIG. 9 is a comparison diagram of an actual lateral image that is generated through the first and second deep learning processes and a prediction image, with respect to a lateral image of a spine photographed in the C-arm equipment.

FIG. 10 is a construction diagram of trocar in which a position sensor and a pressure sensor are installed according to an embodiment of the present disclosure.

FIG. 11 is an operating state diagram of the trocar illustrated in FIG. 10.

FIG. 12 is a schematic cross-sectional view illustrating components according to another embodiment of a trocar handle, that is, a main part of the present disclosure.

FIG. 13 is a cross-sectional diagram illustrating detailed components of the trocar handle illustrated in FIG. 12.

FIG. 14 is a side view of a trocar handle, that is, a main part of the present disclosure.

FIG. 15 is an appearance view of a PCB substrate embedded in the trocar handle, that is, a main part of the present disclosure.

FIG. 16 is a circuit block diagram illustrating detailed components of the PCB substrate, that is, a main part of the present disclosure.

FIG. 17 is an operating diagram of the pressure sensor for describing a process of pressure being changed by hammering on the trocar handle, that is, a main part of the present disclosure.

FIGS. 18A and 18B are processing flowcharts illustrating a real-time surgery support method using the sensor-based AI segmentation model according to an embodiment of the present disclosure.

FIG. 19 is a processing flowchart illustrating a learning process of the AI segmentation model, that is, a main part of the present disclosure.

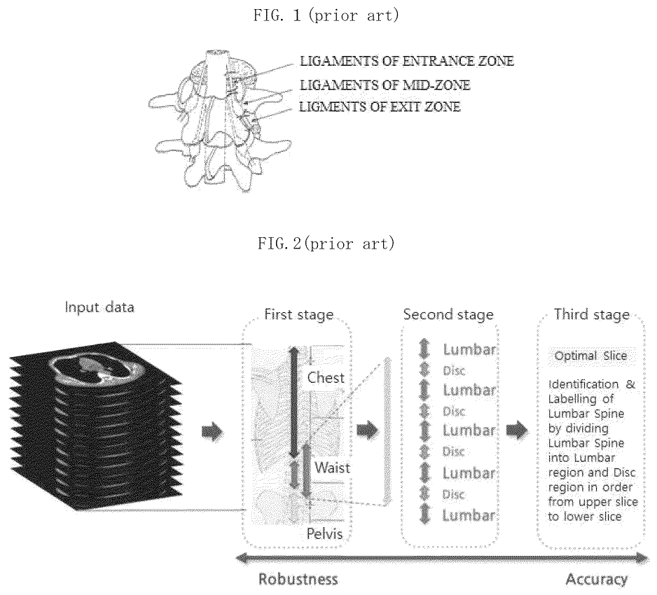

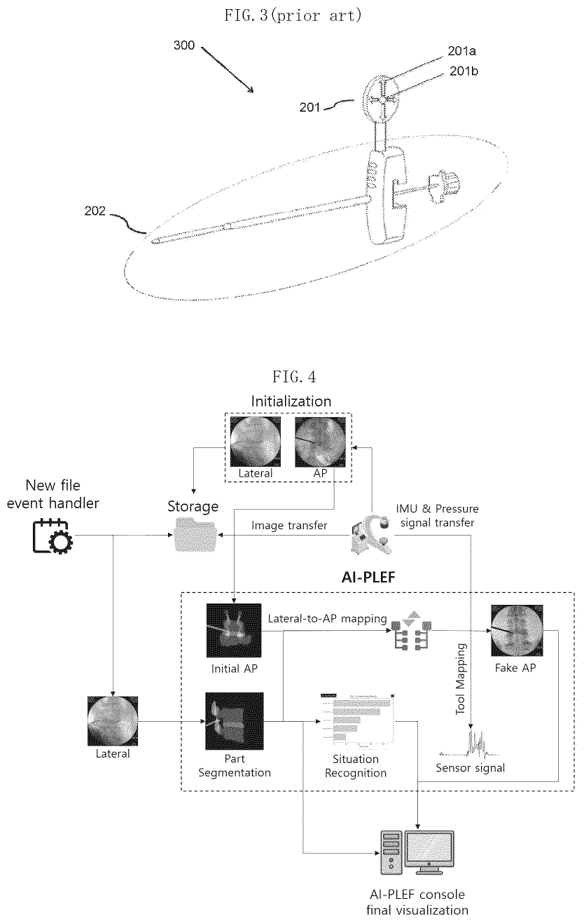

### DETAILED DESCRIPTION

Hereinafter, embodiments of the present disclosure are described in detail with reference to FIGS. 4 to 19.

A real-time surgery support method using a sensor-based AI segmentation model and a computer system for performing the real-time surgery support method according to embodiments of the present disclosure have been embodied to be capable of supporting a real-time surgical process of a surgical operator by generating a lateral image of a spine from an AP image of the spine photographed in C-arm equipment and using an integrated image of the AP image and the lateral image with which position and pressure data of the surgical instrument are overlaid.

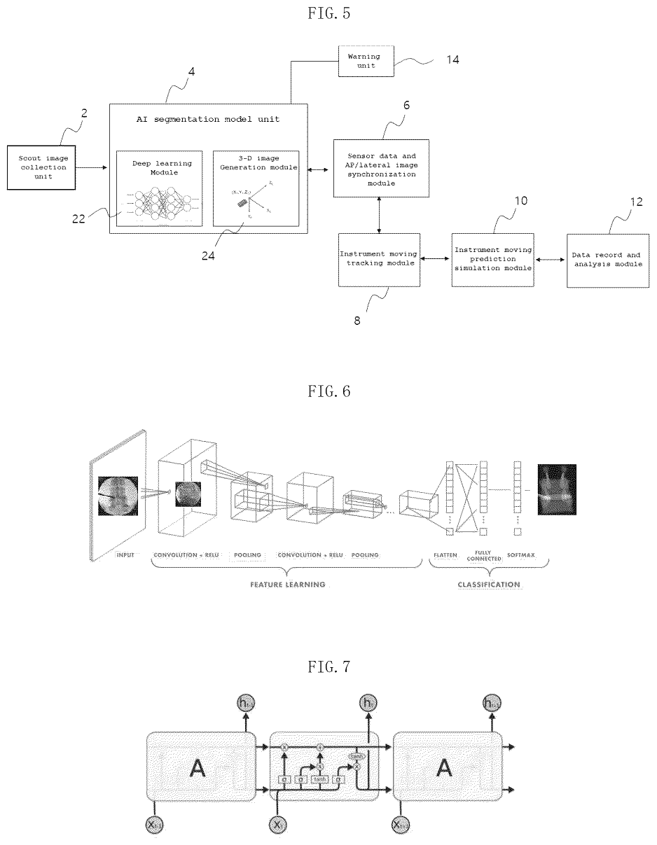

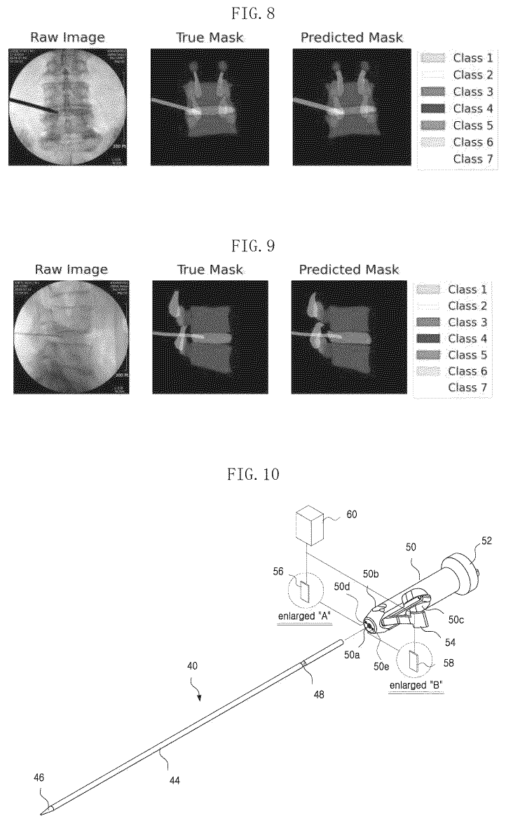

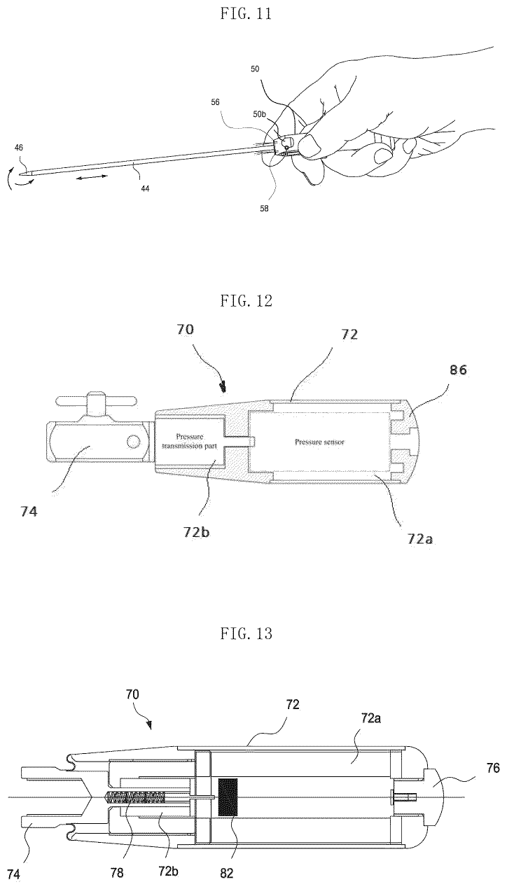

FIG. 4 is a schematic diagram illustrating a computer system for supporting a real-time surgery using a sensor-based AI segmentation model according to an embodiment of the present disclosure. FIG. 5 is a block diagram illustrating components of the computer system for supporting a real-time surgery using the sensor-based AI segmentation model according to an embodiment of the present disclosure. FIG. 6 is a concept view of a first deep learning process of the AI segmentation model, that is, a main part of the present disclosure. FIG. 7 is a concept view of a second deep learning process of the AI segmentation model, that is, a main part of the present disclosure. FIG. 8 is a comparison diagram of an actual AP image that is generated through the first and second deep learning processes and a prediction image, with respect to an AP image of a spine photographed in C-arm equipment. FIG. 9 is a comparison diagram of an actual lateral image that is generated through the first and second deep learning processes and a prediction image, with respect to a lateral image of a spine photographed in the C-arm equipment. FIG. 10 is a construction diagram of trocar in which a position sensor and a pressure sensor are installed according to an embodiment of the present disclosure. FIG. 11 is an operating state diagram of the trocar illustrated in FIG. 10.

As illustrated in FIG. 4, according to an embodiment of the present disclosure, initial images (i.e., an AP image and lateral image) of an intervertebral foramen region are obtained by a new file event handler and input to storage in a surgical process. An AP image and a lateral image that are input to C-arm equipment in real time are invoked from an AI-PLEF console, processed and transformed into images, and input to the storage. The new file event handler produces a part segmentation image by labelling the lateral image for each region of an anatomical structure of the intervertebral foramen. The initial AP image and the lateral part segmentation image are stored in an image recognition unit (situation recognition). Furthermore, after a fake AP image is generated by mapping the AP image and the lateral image in the AI-PLEF console, the fake AP image is stored in the image recognition unit. In particular, the AI-PLEF console generates the lateral image (or the AP image) based on only the AP image (or the lateral image) by using a mathematical formula and a 3-D transformation method. The AI-PLEF console maps the AP image (or the lateral image) photographed in the C-arm equipment and a surgical instrument, and performs tracking in real time by detecting a moving signal. An AP image (or a lateral image) and an image of the surgical instrument are generated together through deep learning of the AI-PLEF console, thereby improving the accuracy of a surgery while tracking a moving process of surgical instrument.

In an embodiment of the present disclosure, the entire process of generating a lateral image (or an AP image) from an AP image (or a lateral image) and tracking a moving process of surgical instrument is stored and visualized in the AI-PLEF console.

In an embodiment of the present disclosure, a 3-D transformation method refers to a method of generating two lateral images and a plan image (or a plan image and a lateral image) based on only one plan (or lateral) image. A term “3-D” should be interpreted as the same meaning as that described above.

Detailed components of a computer system according to an embodiment of the present disclosure by which the series of processes are performed are described in detail with reference to FIG. 5.

In an embodiment of the present disclosure, as illustrated in FIG. 5, the computer system includes a scout image collection unit 2 configured to collect an anteroposterior (AP) image and lateral image of an anatomical structure of a spine by using C-arm equipment in order to perform supervised learning, an AI segmentation model unit 4 including a deep learning module 22 configured to generate a two-dimensional (2-D) image by learning the AP and lateral images collected by the scout image collection unit 2 and a three-dimensional (3-D) image generation module 24 configured to generate a lateral image (or an AP image) from the AP image (or the lateral image) by applying a mathematical formula model, a 3-D transformation model, and a classification/minimum dimension model, a sensor data and AP/lateral image synchronization module 6 configured to synchronize the AP/lateral images generated by the AI segmentation model unit 4 and the data of the position sensor and the pressure sensor mounted on a trocar 40, an instrument moving tracking module 8 configured to visualize the data of the position sensor and the pressure sensor synchronized with the AP/lateral images by tracking the data of the position sensor and the pressure sensor, an instrument moving prediction simulation module 10 configured to predict and simulate a movement of instrument that moves in the instrument moving tracking module 8 in real time, and a data record and analysis module 12 configured to store and analyze data generated by the instrument moving prediction simulation module 10.

In an embodiment of the present disclosure, the scout image collection unit 2 is prepared to collect an AP image and lateral image of an actual spine from the C-arm equipment so that AI can segment an initial anatomical structure of the spine. The scout image collection unit 2 segments the region of the anatomical structure of the spine by performing a labelling task of dividing major surgical regions, that is, a vertebral body, intervertebral foramen, a facet line, nerve, and ligaments in the AP image of the spine, which is obtained by the C-arm equipment. In a labelling process according to an embodiment of the present disclosure, specific structures, such as a superior articular process, a facet joint capsule, and an inferior articular process, are divided so that they can be identified in the scout image collection unit 2 in advance, thereby generating an accurate segmentation model for the preparation of a surgery. In an embodiment of the present disclosure, the labelling of the scout image is to transform a medical term that is used in a surgical process and a surgery step into data by coding the medical term and the surgery step.

In this case, the label is explicit information that is used to classify and identify data, and refers to a category or class to which data belongs. For example, assuming that photograph data of a dog and a cat are present, information indicating whether the data are of a dog or a cat corresponds to a label. The label is mainly used in a classification model of a supervised learning method for machine learning or deep learning. The classification model uses a label as answer data for an output value after receiving data. As described above, the scout image collection unit 2 segments and recognizes the region of the anatomical structure of the spine in advance by performing a labelling task on the scout image. A surgical operator can perform a surgery while confirming an accurate position of surgical instrument that moves in AP and the lateral images in real time.

The scout image collection unit 2 compares a segmentation image generated from the scout image and the AP and lateral images processed by the AI segmentation model unit 4 in real time. Accordingly, accuracy can be improved, and the location of the surgical instrument to be described later can be tracked in real time in the AP and lateral images.

The deep learning module 22 of the AI segmentation model unit 4 learns scout AP/lateral images in order to generate the scout AP/lateral images as a 2-D image. As illustrated in FIG. 6, when an AP image is input to an input stage INPUT, the deep learning module 22 segments anatomical structures for each region in the AP image, and labels and learns the anatomical structures.

The AI segmentation model unit 4 may further include a warning unit 14 configured to predict a movement of the surgical instrument in the AP and lateral images and to generate a warning signal to a surgical operator when the surgical instrument deviates a safe region. The warning unit 14 performs a function for tracking the position of the surgical instrument in the AP and lateral images based on initial segmentation data obtained from the scout image and automatically giving warning when the surgical instrument approaches a danger area or comes into contact with a dangerous tissue. The warning unit 14 receives a position signal and pressure signal that are generated by detecting the position sensor and the pressure sensor to be described later, and generates a warning sound when the surgical instrument deviates a segmentation region of intervertebral foramen.

In an embodiment of the present disclosure, the AI segmentation model unit 4 may further include a data feedback system (not illustrated) configured to update AI segmentation in real time by incorporating a change in AP and lateral images while a surgery is in progress. The data feedback system performs a function for maintaining a segmentation model generated from a scout image in the latest state by incorporating an anatomic change or a change in the position of instrument, which occurs during surgery, in real time.

In an embodiment of a deep learning model according to an embodiment of the present disclosure, the segmentation region of intervertebral foramen is classified and labelled by applying a convolutional neural network (CNN) model, such as that illustrated in FIG. 6, and a long short-term memory (LSTM) model, such as that illustrated in FIG. 7.

As illustrated in FIG. 6, the CNN model includes an input layer, a hidden layer, and an output layer. Each of the input layer, the hidden layer, and the output layer learns unique features, by including a convolution layer, an active or ReLu layer, and a pooling layer, and performing learning.

A data set that is learnt through a CNN learning algorithm is processed through an LSTM algorithm. As illustrated in FIG. 7, the LSTM model consists of an input gate, a forget gate, an output gate, and a cell state. The LSTM model determines only data to be memorized through the forget gate, among input data, as a next output value. That is, labelling generated by the CNN algorithm derives a similar image as an output value in the LSTM algorithm.

It has been known that a segmentation region may be classified in the anatomical structure of intervertebral foramen by using a U-Net model and a mask recurrent convolution product neural network (mask R-CNN) model as another deep learning model capable of replacing the CNN model in order to learn an AP or lateral image of the C-arm equipment.

The 3-D image generation module 24 of the AI segmentation model unit 4 generates a lateral segmentation image from the AP image (or lateral image) of the C-arm equipment by using the mathematical formula and a 3-D transformation vector. The 3-D image generation module 24 recognizes the anatomical structure of an AP image and a lateral image that have been learnt through a deep learning model. In a process of recognizing the anatomical structure, a data labelling method, a data pre-processing technology, and a specific labelling rule are set.

Furthermore, the 3-D image generation module 24 completes a scout segmentation image in which AP and lateral images have been integrated by providing guidance to the AP and lateral images collected by the scout image collection unit 2 so that AP and lateral images generated through self-learning are overlaid with the AP and lateral images.

Furthermore, the 3-D image generation module 24 recognizes the completed scout segmentation image as supervised learning data, and generates a lateral image (or an AP image) from an AP image (or a lateral image) by using the mathematical formula and the 3-D transformation model.

A learning method of generating, by the 3-D image generation module 24, a lateral image (or an AP image) from an AP image (or a lateral image) is as follows.

First, the 3-D image generation module 24 learns 3-D space understanding through the pair of two views (i.e., AP and lateral views). When AP image is provided through a deep learning model, the 3-D image generation module 24 predicts and generates a lateral image through the accurate recognition of a landmark and the calculation of a 3-D mathematical transformation model. That is, in order to reproduce a lateral image changed into an angle of 90°, the 3-D image generation module 24 transforms 2-D coordinates into 3-D space coordinates. To this end, the 3-D image generation module 24 calculates the 3-D coordinates by applying a rotation matrix, that is, a geometric transformation method. That is, the 3-D image generation module 24 transforms the coordinates of an AP image into the coordinates of a lateral image by applying a rotation matrix.

When the coordinates obtained from the AP image are laterally transformed, the coordinates are rotated 90° around a y axis.

$$R = [ \begin{matrix}cos ( θ ) & 0 & sin ( θ ) \\ 0 & 1 & 0 \\ - sin ( θ ) & 0 & cos ( θ )\end{matrix} ] \qquad [ Rotation matrix ]$$

In this case, since θ=−90°,

$$R = [ \begin{matrix}0 & 0 & 1 \\ 0 & 1 & 0 \\ - 1 & 0 & 0\end{matrix} ]$$

is obtained.

Accordingly, the coordinates (x, y) of the AP image are transformed into the coordinates (x′, y′, z′) of the lateral image.

After AP coordinates (xAP and yAP) are expanded to 3-D coordinates (xAP, 0, and zAP), if the rotation matrix is applied,

$$[ \begin{matrix}{x}_{LAT} \\ {y}_{LAT} \\ {z}_{LAT}\end{matrix} ] = R \cdot [ \begin{matrix}{x}_{AP} \\ {y}_{AP} \\ 0\end{matrix} ]$$

is obtained. As a result,

$$[ \begin{matrix}{x}_{LAT} \\ {y}_{LAT} \\ {z}_{LAT}\end{matrix} ] = [ \begin{matrix}0 \\ {y}_{AP} \\ - {x}_{AP}\end{matrix} ]$$

is obtained.

Accordingly, a movement in the AP image is laterally transformed into zLAT=−xAP, yLAT=yAP.

Through the AI segmentation model unit 4, a surgical operator can accurately recognize the position of a facet line and an anatomical structure, while watching the overlaid AP/lateral images, and can be provided with guidance to a moving path of the instrument in real time.

As described above, when the lateral image is generated from the AP image through the 3-D image generation module 24, as illustrated in a process of transforming an AP image into an image in FIG. 8, and a process of transforming a lateral image in to an image in FIG. 9, accuracy is checked by comparing a true mask and a prediction mask based on the raw data of actual AP and lateral images, and accuracy and reproducibility are improved through a consistent learning process.

The sensor data and AP/lateral image synchronization module 6 synchronizes the coordinates of surgical instrument and AP and lateral images so that the AP and lateral images are overlaid with the coordinates of the surgical instrument, in order to provide accurate position information by tracking only the movement of sensor data in real time in the state in which the AP and lateral images have been set as a fixed background. In this case, the sensor data include coordinate data of the position sensor and force pattern data of the pressure sensor.

The sensor data and AP/lateral image synchronization module 6 maintains consistency between AP/lateral images through a synchronization algorithm that associates the coordinates of surgical instrument with an AP image and a lateral image, and recognizes and distinguishes between an important anatomical structure and the surgical instrument in the AP image and the lateral image. In this case, the sensor data and AP/lateral image synchronization module 6 uses a pre-learnt data set and labelling so that the tissues of the anatomical structure can be accurately recognized. In particular, the sensor data and AP/lateral image synchronization module 6 has been designed to distinguish between a spine and surrounding tissues and update the tissues and a movement of the trocar 40 in real time. Accordingly, a surgical operator can confirm the same anatomical structure at all of angles, and can consistently adjust a movement of the trocar 40. A surgical operator can confirm an accurate position of instrument in two directions (i.e., AP and lateral) through AP/lateral segmentation images, and can precisely adjust the position of the trocar 40.

An embodiment of the present disclosure proposes an example in which a position sensor 56 and a pressure sensor 58 are mounted on the instrument of the trocar 40 that is used in PLEF.

The trocar 40 percutaneously enters a spine facet joint based on the guidance of a guide tube (not illustrated), reaches a transforaminal ligament's resecting target point of intervertebral foramen, and performs a function for providing guidance to the entry of surgical instruments and also primarily resecting a target ligament. The trocar 40 includes a needle 44 including a groove 48 formed in a predetermined part thereof and a long and thin pole having a pointed needle tip 46 at a front end thereof, a handle 50 into which the needle 44 is inserted and which can apply a force so that directionality, straightness, and steerability are easy based on a percutaneous incident angle of a spinal facet joint, the position sensor 56 installed on one side of the handle 50 and configured to generate coordinate data, and the pressure sensor 58 installed on the other side of the handle 50 and configured to generate a force pattern when hammering that strikes surgical instrument is applied.

The handle 50 includes a groove 50a formed in a central part thereof so that the needle 44 is inserted into the groove, a direction indication surface 50b formed of a flat surface so that the direction indication surface becomes a reference for an entry angle at the top of the front end side of the handle, an accommodation space 50c caved in one side of the handle at a predetermined depth, and buried grooves 50d and 50e formed on both sides of the groove 50a and having the position sensor 56 and the pressure sensor 58 buried therein, respectively. Furthermore, the position sensor 56 and pressure sensor 58 of the trocar include a mapping module 60 configured to receive coordinate data and force pattern data and to map the coordinate data and force pattern data to AP and lateral images. In general, the mapping module 60 may include a computer main body, a monitor, and a keyboard. The keyboard performs a function for generating a stop/release signal for setting the reference coordinates of a surgery path in order to change the reference coordinates based on a movement of the position sensor 56 and the pressure sensor 58.

As illustrated in FIG. 11, the trocar constructed as described above reaches a target point as a surgical operator percutaneously inserts the needle 44 into intervertebral foramen and moves the needle 44 up and down with the handle in his or her hand. At this time, as coordinates generated by the position sensor 56 are mapped to a lateral image, the surgical operator can confirm the position of the trocar through the monitor.

The instrument moving tracking module 8 tracks an incident angle, a moving distance, and a moving direction based on the coordinates of the position sensor 56, which have been mapped to an AP image and a lateral image. That is, the instrument moving tracking module 8 provides guidance to a target point from which ligaments will be removed by tracking a movement of the trocar 40 in the AP/lateral images.

Another embodiment of the trocar, that is, a main part of the present disclosure, is described with reference to FIGS. 12 to 17.

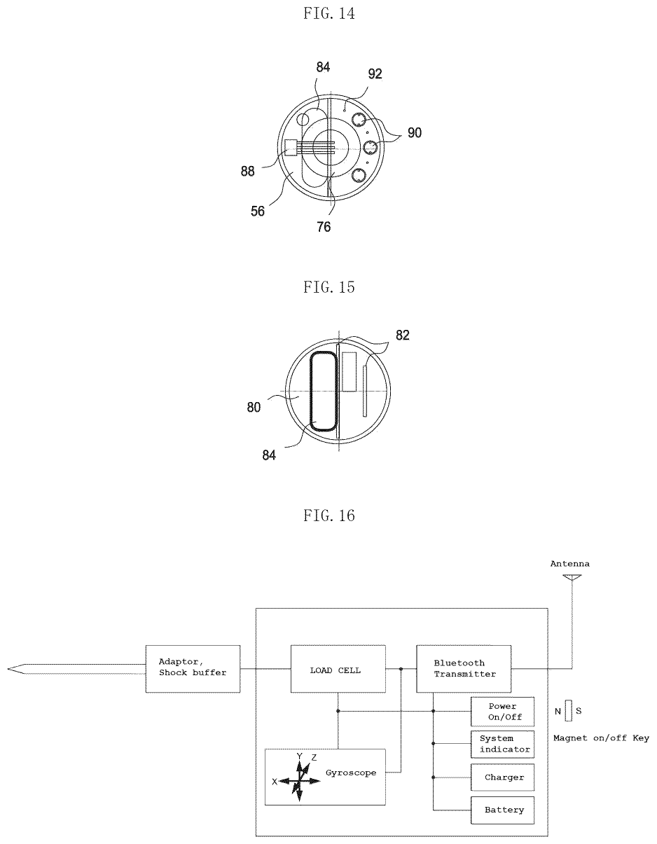

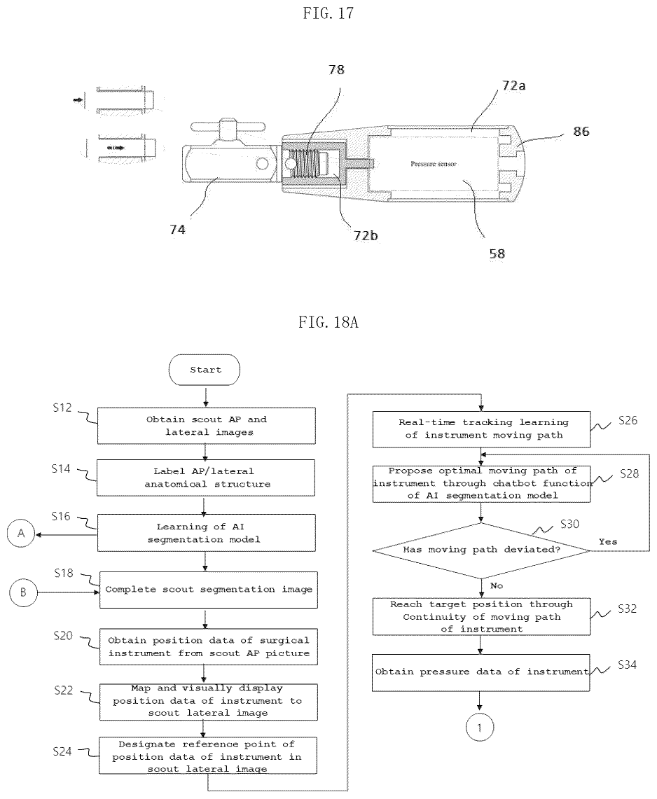

FIG. 12 is a schematic cross-sectional view illustrating components according to another embodiment of a trocar handle, that is, a main part of the present disclosure. FIG. 13 is a cross-sectional diagram illustrating detailed components of the trocar handle illustrated in FIG. 12. FIG. 14 is a side view of a trocar handle, that is, a main part of the present disclosure. FIG. 15 is an appearance view of a PCB substrate embedded in the trocar handle, that is, a main part of the present disclosure. FIG. 16 is a circuit block diagram illustrating detailed components of the PCB substrate, that is, a main part of the present disclosure. FIG. 17 is an operating diagram of the pressure sensor for describing a process of pressure being changed by hammering on the trocar handle, that is, a main part of the present disclosure. In these drawings, the same components as those described with reference to FIGS. 10 and 11 are assigned the same reference numerals.

As illustrated in FIG. 12, a trocar handle 70 includes a cylinder 72 having two space parts 72a and 72b, an adaptor 74 coupled to one side of the cylinder 72 and having the needle 44 inserted therein, and a hammer anvil 76 installed on a cross section of the cylinder 72 and capable of applying hammering to a tissue in intervertebral foramen.

As illustrated in FIG. 13, a pressure sensor 58 is installed in a space part 72a on one side of the cylinder 72. A space part 72b on the other side of the cylinder 72 is a pressure transfer space. A shock buffer 78 is installed in the pressure transfer space 72b, reduces strength that applies striking pressure of hammering to the pressure sensor 58, and generates a signal. In an embodiment of the present disclosure, the shock buffer may be formed of a spring.

As illustrated in FIG. 15, a sensor connector 80 is mounted on the space part 72a on the other side of the cylinder. A circuit board 82 and a battery 84 are embedded in the sensor connector 80.

As illustrated in FIGS. 13 and 14, a cover 86 is installed at the end of the space on one side of the cylinder 72. A position sensor 56, a power switch 88, LED lamps 90 for indicating a charging state, and an antenna 92 for communicating with the AI segmentation model are mounted on the cover 86.

In the trocar handle 70 constructed as above, the adaptor 74 and the pressure sensor 58 are disposed at a predetermined interval without coming into direct contact with each other, as illustrated in FIG. 17. The interval functions to collect pressure and transfer the pressure to the needle 44, when striking for hammering is applied to the cylinder. That is, as indicated by an arrow in FIG. 17, when hammering is applied, pressure of the needle is transferred to the shock buffer 78 and transferred to the pressure sensor 44 in the state in which the pressure has been reduced to some extent. Accordingly, the pressure sensor 44 generates a pressure signal.

In an embodiment of the present disclosure, the position sensor 56 may be formed of a load cell and a gyroscope. As illustrated in the circuit configuration illustrated in the PCB substrate of FIG. 16, the trocar 40 according to an embodiment of the present disclosure is connected to the shock buffer 78 through the adaptor 74. The load cell and the gyroscope for generating position coordinates are disposed in the PCB substrate. The position coordinates generated by the load cell and the gyroscope are applied to the antenna through a Bluetooth transmitter, and are transmitted to the mapping module 60. Circuit patterns for connecting the load cell, the gyroscope, the power (on/off) switch, the system indicator, the charger, and the battery are formed on the PCB substrate.

A signal that is generated through the trocar handle 70, the position sensor 56, and the pressure sensor 58 is transmitted to the mapping module 60. Furthermore, a movement of the needle 44 is stopped or released by a stop (on)/release (off) operation of the enter key of the keyboard. A stop point of the needle 44 becomes a coordinate reference point of the position sensor 56 and is used to measure an angle and moving length of a movement of the trocar at AP and lateral positions.

Referring back to FIG. 5, the instrument moving tracking module 8 improves the accuracy of an image changeover (i.e., generate two AP/lateral images based on one AP image (or lateral image)) of the 3-D image generation module 24 by combining the aforementioned various learning models, tracks a movement of the trocar 40 in real time, and provides fast feedback during surgery.

The instrument moving tracking module 8 generates a lateral image in real time based on the coordinates of an AP image and a movement pattern of instrument. In this case, a deep learning model, such as a long short-term memory (LSTM) model or a convolutional neural network (CNN) model, may be used to predict a next frame based on information of a previous frame. The deep learning model feeds back an error by comparing a predicted lateral image and an actual lateral image. Accordingly, the deep learning model can be trained more precisely.

In this case, the LSTM model predicts the position of a next frame based on information of a previous frame by processing a movement of instrument and sensor data in a time-series way. Accordingly, 3-D coordinates can be predicted more accurately.

The CNN model recognizes the anatomical structure of an image of the C-arm equipment and the position of surgical instrument, and visualize an incident angle and moving distance of the trocar 40 based on the anatomical structure and the position of the surgical instrument. The mathematical model may derive information on the insertion depth of the trocar 40 in a lateral image by calculating the angle and distance of the trocar 40 based on position data extracted by the CNN model. In this process, accurate 3-D coordinates may be calculated through a trigonometric function and an interpolation method. Furthermore, the LSTM model and the CNN model may be integrated to transform the predicted 3-D coordinates into a lateral image. The lateral image may be synchronized with an AP image and then provided to a surgical operator in real time. Accordingly, the surgical operator can check AP and lateral images at the same time and track a real-time movement of the trocar 40.

The generation of a lateral segmentation image (or AP image) from an AP image (or a lateral image) by the instrument moving tracking module 8 is performed by labelling information, such as the moving path, angle, and position of instrument and constructing a data set. The data includes various surgical examples, and help the instrument moving tracking module 8 to accurately recognize and predict the position of surgical instrument even in various situations. The instrument moving tracking module 8 learns a moving pattern and movement of the surgical instrument based on the labelled data set. Accordingly, where the surgical instrument moves from a location at a certain angle and a moving distance of the surgical instrument can be predicted in real time. A lateral image can be generated based on only one AP image of the C-arm equipment in real time. Accordingly, the 3-D structure of a surgical part can be checked in real time based on only the AP image (or the lateral image) even without photographing an additional lateral image.

The instrument moving prediction simulation module 10 reconstructs a lateral image based on the data of an AP image through the mathematical model, and displays that the position and movement of instrument are synchronized in real time.

If deep learning through labelling data is performed through such a method, AP and lateral images can be transformed based on only AP (or lateral) raw data photographed by the C-arm equipment, and the accurate tracking of a movement of instrument can be simulated. Accordingly, a surgical operator can perform a surgery while receiving more reliable feedback, and can perform a surgery with higher accuracy while reducing the photographing of an additional image using the C-arm equipment during the surgery.

In an embodiment of the present disclosure, a lateral (or AP) image has been generated from an AP image (or lateral image) of the C-arm equipment, and a process of mapping AP and lateral images has been visualized by using the LSTM and CNN models, but the present disclosure is not limited thereto. One or more learning models selected, among a transformer model, 3-D convolutional neural networks (3-D CNNs), a recurrent convolutional neural network (RCNN), generative adversarial networks (GANs), attention mechanisms, autoencoders, and reinforced learning, may be applied.

A transformer-based vision transformer (ViT) is a model that learns important features in an image by processing an image in a patch unit. The transformer can improve the accuracy of recognizing an anatomical structure in a complicated 2-D-3-D changeover task. Furthermore, the transformer can effectively learn temporal dependency that is more complicated than the LSTM model, and may help better understanding of a temporal correlation between sensor data and a frame of an image of the C-arm equipment.

The 3-D CNNs are a learning model that has been optimized to learn 3-D data (e.g., an image of several frames), and can more accurately learn depth and space information of a 2-D image for an AP-lateral changeover in an image of the C-arm equipment. Furthermore, the 3-D CNNs may be used to analyze time-series data because the 3-D CNNs can process an image changeover according to a time flow by inputting continuous frames in a 3-D way and accurately track a moving path of the surgical instrument in a fixed image.

The RCNN is a model in which the spatial feature learning ability of the CNN model and the temporal sequence learning ability of the LSTM model are combined, and has an advantage in analyzing a movement of surgical instrument in a time-series way while recognizing an anatomical structure within an image. In particular, the RCNN can more precisely track an interaction between a position change of instrument in a real-time feedback system and an anatomical structure.

The GANs are a learning model capable of visualizing a lateral image more sharply and accurately by estimating the lateral image in an AP image because the GANs have an excellent image generation ability, and enables data augmentation through non-supervised learning. That is, if the GANs are used, learning can be reinforced by generating various forms of virtual data in a situation in actual surgery data are insufficient.

The attention mechanism is a model that helps AI to concentrate on an important part of an anatomical structure, and can improve the accuracy of a 2-D-3-D changeover by concentrating on a facet line and an important structure around intervertebral foramen, for example. Furthermore, real-time feedback can be provided more rapidly and accurately during surgery because necessary information can be focused depending on a position change of instrument through the attention mechanism.

The autoencoder is a learning model that is used to extract only important features by compressing image data. This model may be used to efficiently process the image data of high-resolution C-arm equipment and to leave only important anatomical features. The autoencoder may be used to learn various images that are generated during surgery and to extract only pre-processed important features, and may use the extracted important features as information necessary for a real-time 2-D-3-D changeover.

Reinforced learning is a learning model that is used to enable AI to adjust a movement of instrument in real time during surgery and to transmit feedback, and enables AI to optimize an optimal position and movement of surgical instrument while learning the optimal position and movement in real time. Furthermore, the reinforced learning may train AI to dynamically handle various surgery situations and enable AI to learn and apply an optimal path of a movement of surgical instrument in various situations.

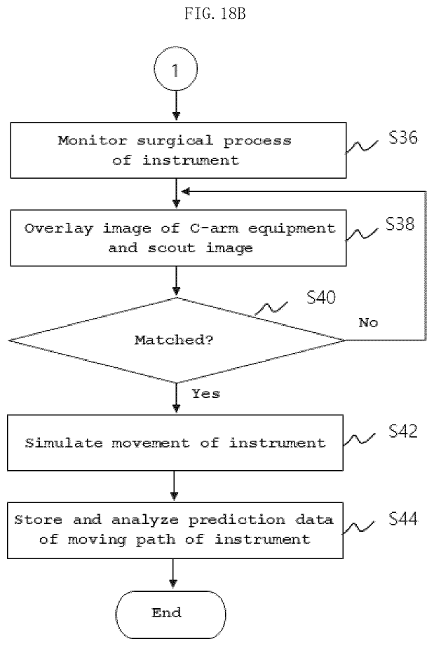

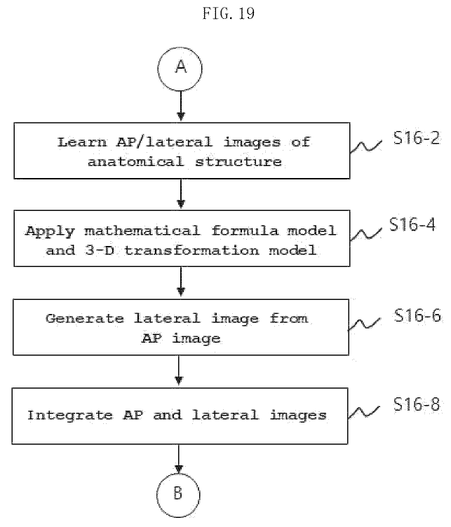

A real-time surgical support method using a sensor-based AI segmentation model, which has been constructed as above, according to an embodiment of the present disclosure, is described with reference to FIGS. 18 and 19.

FIGS. 18A and 18B are processing flowcharts illustrating a real-time surgery support method using the sensor-based AI segmentation model according to an embodiment of the present disclosure. FIG. 19 is a processing flowchart illustrating a learning process of the AI segmentation model, that is, a main part of the present disclosure.

As illustrated in the drawings, AP and lateral images of a spine are collected from the C-arm equipment (S12). The AP and lateral images are basic scout images for supervised learning and are targets of learning. Next, each tissue region of an anatomical structure is classified and labelled by pre-processing the AP/lateral images (S14). AP and lateral images are obtained from the labelled AP and lateral images through deep learning of the AI segmentation model (S16).

As illustrated in FIG. 19, the AI segmentation model learns the AP/lateral images of the anatomical structure by using the CNN algorithm and the LSTM algorithm that are included in the deep learning module 22 (S16-2). The 3-D image generation module 24 learns the generated AP image and lateral image, and generates a lateral image (or an AP image) from the AP image (or the lateral image) by using the mathematical formula algorithm and the 3-D transformation algorithm (S16-4 and S16-6). Thereafter, a surgical operator can view the AP image and the lateral image on one screen by integrating the AP image and the lateral image (S16-8). A scout segmentation image is completed by integrating the AP image and the lateral image (S18).

Next, the position data of surgical instrument are obtained from a scout AP image that is initially collected (S20). Furthermore, the position data of instrument are mapped to a scout lateral image, visualized and displayed on the monitor (S22).

A reference point of the position data of the surgical instrument is designated in the scout lateral image (S24). For example, the reference point may be a hammer point. The reference point of the position data may be designated up to 4 or 5 reference points, such as a facet line and a swing position, within the anatomical structure.

The reference point of the position data is recognized as a landmark. A moving distance of the trocar 40 is calculated on the basis of the landmark. Furthermore, the real-time tracking of a moving path of the surgical instrument is learned (S26). In this case, a process of tracking the moving of the surgical instrument is described as follows.

The position sensor 56 installed in the handle 50 of the trocar 40 functions to generate the coordinated data of an incident angle of the trocar. The position data generated by the position sensor are incorporated into the AP and lateral segmentation images. A real-time coordinate value detected by the position sensor 56 is converted into a 3-D coordinate value, and thus the incident angle and moving distance of the instrument are displayed in the AP and lateral segmentation images. The coordinate data of the position sensor are matched with the AP/lateral images, and the visualization is supported so that the path along which the trocar 40 moves can be seen.

The pressure sensor 58 detects pressure of the instrument which is applied to a specific tissue. When excessive pressure occurs, the pressure sensor 58 provides feedback for the excessive pressure to a surgical operator in real time. For example, when excessive pressure is applied to a nerve or another important tissue, the AI segmentation model automatically gives warning and adjusts a movement of the instrument.

In order to accurately track a movement of the instrument based on the data of the position sensor 56 and the data of the pressure sensor 58, it is important for the AI segmentation model to understand and learn a process by labelling the movement of the instrument in a coded language. To this end, the movement of the instrument may be expressed in detail by using a language system coded as follows.
1. Designate position of instrument (Insertion Point):
Code: Insert_Point
Description: a position into which the instrument is first inserted is designated, and an accurate insertion point confirmed through an image of the C-arm equipment is labelled as a reference.
2. Insertion Depth:
Code: Depth_Level_X (X is classified as an inserted depth level)
Description: a depth at which the instrument is inserted is expressed for each level in a needling process. For example, Depth_Level_1 to Depth_Level_5 are set, and a progress situation for each level is clarified.
3. Angle Adjustment:
Code: Angle_Adjust_Left/Right_X (X is the unit of an angle)
Description: when the instrument is adjusted in a specific direction, the left or right and an angle unit are added and recorded in detail, if necessary, by incorporating the adjustment of the instrument.
4. Advance Check:
Code: Advance_Xmm
Description: when the instrument is moved at a predetermined interval (e.g., a mm unit), a detailed moving distance is labelled by using a method, such as Advance 2 mm.
5. Resistance Noted:
Code: Resistance_Level_X
Description: in a section in which resistance is detected, the strength of the resistance is labelled for each level so that AI can recognize such a section and use the recognition for a decision in the future. For example, Resistance_Level_1 to Resistance_Level_3 may be set.
6. Position Confirmed/Hold Position:
Code: Confirm_Position or Hold_Position
Description: this code is used when the instrument reaches a specific position or when the position of the instrument needs to be fixed. Such labelling enables Al to perform learning by incorporating the state in which a movement of the instrument has been stopped or the instrument has been fixed.
7. Withdraw:
Code: Withdraw_Xmm
Description: when the instrument is withdrawn, if necessary, a withdrawn distance (e.g., mm) is specified and recorded. For example, the withdrawn distance may be expressed like Withdraw_2 mm.
8. Vibration or Oscillation:
Code: Oscillate_X (X is vibration strength)
Description: this code is used to accelerate the insertion of the instrument by finely vibrating the instrument, if necessary. The strength of vibration may be divided for each step like Oscillate_1.

The labelling system records a moving and manipulation of the instrument in a detailed and consistent way so that the AI segmentation model unit 4 can clearly learn a needling process. Furthermore, the labelling system can contribute to the improvement of future prediction and real-time feedback functions because the AI segmentation model unit 4 analyzes a moving pattern and process of Tuohy needling based on labelled data. Furthermore, when resistance or a significant abnormality, such as a position change, occurs, the labelling system enables the AI segmentation model unit 4 to rapidly recognize a situation and to provide necessary feedback to a surgical operator.

If a movement of the instrument is labelled in the coded language, Al can learn a precise surgical process without a sensor even in a Tuohy needling process, and a base on which subsequent feedback can be provided can be prepared.

A coding system for such a movement of the instrument adds ‘up and down’ information so that the AI segmentation model unit 4 can easily confirm the incident angle and direction of the instrument. Accordingly, an intuitive direction of the instrument can be presented because a moving direction of the instrument can be presented more clearly rather than using only numbers. Furthermore, the learning of AI and the accuracy of feedback are improved because a specific position or an anatomical structure are recognized in numbers and guidance can be provided to adjust an angle by a direction. Furthermore, immediate measures can be taken in a situation in which the angle or position of the instrument need to be adjusted because AI can analyze both numbers and direction information and provide feedback in real time during surgery.

The coding system in which numbers and direction information are combined as described above helps the AI segmentation model unit 4 to clearly recognize an important point in a surgical process and the adjustment of an angle of the instrument.

If an image of the C-arm equipment is photographed more frequently by increasing the number of times of photographing in a process of capturing a minute movement of the instrument based on sensing signals of the position sensor 56 and the pressure sensor 58, a movement of the instrument can be recorded more finely. Accordingly, the following effects may be expected.

First, the AI segmentation model unit 4 can learn a fine movement and position change of instrument more accurately because a small change in a movement of the instrument or detailed moving path of the instrument can be checked if photographing frequency is high.

Second, if a sequence of continuous images is obtained when instrument is moved in the state in which an interval of photographing has been reduced, the AI segmentation model unit 4 can naturally recognize how a movement of the instrument is connected. Accordingly, the AI segmentation model unit 4 can be helpful to control a movement of the instrument more smoothly and consistently in an actual surgery because the AI segmentation model unit 4 understands a moving pattern and natural flow of the instrument.

Third, if more data are obtained by increasing the number of times of photographing, the AI segmentation model unit 4 can track the position of the surgical instrument more accurately in real time and provide feedback. This may contribute to the improvement of the accuracy and safety of a surgery by consistently monitoring the position of the surgical instrument without missing the position of the surgical instrument.

Fourth, the AI segmentation model unit 4 can deeply learn how to manipulate the surgical instrument in various surgery situation because various angles and speeds of the moving surgical instrument can be captured more finely. Accordingly, this helps AI to make an accurate decision even in an unexpected situation.

Fifth, the ability of the AI segmentation model unit 4 to predict a moving path based on more data is improved if the number of times of photographing is increased. Performance to predict a next position is improved through the learning of a moving pattern. Accordingly, an AI-based robot or an assistant system can be accurately controlled.

In an embodiment of the present disclosure, an incident angle and moving distance of the trocar are transmitted in real time through the position sensor 56 and the pressure sensor 58 mounted on the handle 50 of the trocar 40. Accordingly, the following important advantages in the learning and a surgery assistance of the AI segmentation model unit 4 are provided.

First, the position sensor 56 can precisely measure an angle at which the trocar is inserted. Accordingly, the AI segmentation model unit 4 checks a change in the incident angle of the trocar in real time and provides the trocar with an optimal angle, thereby minimizing damage to a surrounding tissue.

Second, the position sensor 56 tracks a distance in which the trocar moves so that the instrument properly reaches a target point. In particular, this is important in a ligament resecting process. This can prevent the trocar from being inserted too deeply or shallowly, and can assist a task to be performed at a proper position.

Third, the pressure sensor 58 monitors pressure that is applied to the trocar in real time. Accordingly, the AI segmentation model unit 4 can recognize the extent that the trocar comes into contact with a ligament or a bone, and may provide feedback so that pressure can be adjusted, if necessary. This contributes to the improvement of safety during surgery by preventing an excessive force from being applied.

Fourth, the AI segmentation model unit 4 learns an optimal moving pattern and angle of the trocar in various situations by accumulating angle and moving distance data of the trocar. The AI segmentation model unit 4 can predict a movement of the trocar based on accumulated data, and can present a proper moving path of the trocar in real time during surgery.

Fifth, the AI segmentation model unit 4 can calculate the most efficient and safe moving path of the trocar based on position and pressure data that are provided in real time. In particular, the optimization of the moving path of the trocar plays an important role in a complicated resecting task. A surgery can be performed more precisely and consistently through a path predicted by the AI segmentation model unit 4.

Sixth, when a movement of the trocar deviates in an unexpected direction or pressure is suddenly changed, the AI segmentation model unit 4 immediately detects such a movement and pressure and gives warning. Accordingly, the insertion of the trocar in a wrong direction or excessive pressure on the trocar can be prevented.

Through the transfer of such real-time data and the feedback system, the AI segmentation model unit 4 can accurately track a position and movement of the trocar, thereby greatly increasing the accuracy and safety of an AI-PLEF operation.

After step S26 is performed, the AI segmentation model unit 4 proposes an optimal moving path of the instrument to a surgical operator through a chatbot function (S28). In this case, the AI segmentation model unit 4 determines whether the instrument deviates its moving path, generates warning when the instrument deviates a safe region, and functions to automatically correct the moving path, if necessary (S30). Such a warning function restricts the trocar 40 from approaching a sensitive structure area, such as a nerve or a blood vessel, thus protecting the nerve or the blood vessel.

The tracking algorithm of the AI segmentation model unit 4 analyzes data that are generated by the position sensor and the pressure sensor in real time, and provides real-time feedback to a surgical operator so that the position and direction of the instrument can be correctly guided. Through the integration of sensor data and the analysis algorithm, the AI segmentation model unit 4 provides the instrument with guidance so that the instrument can accurately move along a facet line. When the instrument deviates its moving area, the AI segmentation model unit 4 finely adjusts a movement of the instrument and provides the instrument with an optimal position moving path according to an anatomical structure. The integration of sensor data and the analysis algorithm increase the accuracy of the position of the instrument and maximize surgery efficiency.

As described above, the AI segmentation model unit 4 predicts the position of the instrument based on coordinate data of the position sensor 56, and provides a real-time guideline so that the instrument can be adjusted according to a surgery situation. Furthermore, the AI segmentation model unit 4 integrates the data of the position sensor 56 and the pressure sensor 58, immediately recognizes a change in the position and pressure of the instrument by analyzing the integrated data, and supports a surgical operator so that the surgical operator can perform a safe task. For example, when a ligament is resected, the AI segmentation model unit 4 presets the pressure and position of the instrument which is needed when the instrument moves along a facet line, presents the preset pressure and position to a surgical operator, and automatically provides the surgical operator with warning when the instrument deviates the preset pressure and position.

After step S30 is performed, the instrument moving prediction simulation module 10 continuously simulates the moving path of the instrument until the trocar reaches a target position (S32). Furthermore, the instrument moving prediction simulation module 10 obtains force pattern data from the pressure sensor 58 (S34).

In step S32, the position and movement of the instrument are systematically defined by converting the data of the position and pressure sensors in a coded language. For example, pressure, an angle, and a moving direction in a specific position are expressed in the form of a coded command so that AI can structurally understand and use corresponding information. Furthermore, a specific condition related to the position of the instrument can be set through the coded language. Accordingly, a surgical operator can adjust the position of the instrument. For example, a surgical operator can move the instrument to a specific structure through a command, such as “facet approach, 5 mm movement”.

The instrument moving prediction simulation module 10 visualizes an anatomical structure in a 3-D way based on the position and moving data of the instrument so that a surgical operator can confirm the position of the instrument more intuitively.

The instrument moving prediction simulation module 10 accurately calculates the position of the instrument in a 3-D space based on coordinate data obtained from AP and lateral images of the C-arm equipment. Such calculation includes a process of transforming 2-D plan data into 3-D space coordinates based on the mathematical formula. A 3-D model of a surgical part is generated in real time in the AP and lateral images by substituting the coordinate data of the position sensor into the mathematical formula. Through such a function, a surgical operator can confirm an accurate position while watching the 3-D model in addition to a planar 2-D image, and can manipulate the instrument. In particular, a surgical operator can set a surgery path more accurately because a complicated anatomical structure, such as a facet line, a superior articular process, or an inferior articular process, is visualized in a 3-D way.

An embodiment in which 2-D coordinates are converted into 3-D coordinates is as follows.

First, X, Y coordinates of the C-arm equipment are measured in an AP image of equipment. Furthermore, Y, Z coordinates of the same equipment are measured in a lateral image of the C-arm equipment. In this case, consistency between two images is secured because the Y coordinate of the lateral image is used identically with the Y coordinate of the AP image.

Next, 3-D coordinates (X, Y, Z) are calculated by combining the (X, Y) coordinates of the AP image and the (Y, Z) coordinates of the lateral image. For example, a 3-D position P of the equipment is defined as follows.

$$P ( X , Y , Z ) = ( {X}_{AP} , {Y}_{AP} , {Z}_{lateral} )$$

In order to make identical coordinate systems between the two images (i.e., the AP and lateral images), the coordinates are arranged based on a predetermined reference point during surgery. For example, consistency between the two images may be maintained by setting a specific bone structure of a spine or an initial position of instrument as a reference point. In this case, scaling and correction may be performed on the basis of the y axis of the AP and lateral images by using coordinates provided from the data of the position sensor. For example, if the distances of the two images are proportionless, scaling is adjusted based on data provided by the position sensor.

The 3-D coordinates are calculated by combining 2-D coordinates extracted from the AP and lateral images and the data of the position sensor.

The 3-D coordinates (X, Y, Z) are defined as follows.

$$X = {X}_{AP} + Δ {X}_{sensor}$$

$$Y = {Y}_{AP} = YLAT + Δ {Y}_{sensor}$$

$$Z = {Z}_{LAT} + Δ {Z}_{sensor}$$

In this case, ΔXₛₑₙₛₒᵣ, ΔYₛₑₙₛₒᵣ, and ΔZₛₑₙₛₒᵣ are relative position change data with respect to a reference point that is provided by the position sensor. Final 3-D coordinates are calculated by adding the relative position change data to the 2-D coordinates.

An error may occur in the position of the instrument on the basis of the data of the position sensor whenever the instrument moves in a 3-D model, so that the position needs to be corrected in real time. In this case, if the position greatly deviates the reference point, the position is adjusted to an accurate position again based on the data of the position sensor. The position sensor detects a fine movement of the instrument and immediately corrects 3-D coordinates according to a position change. For example, when the instrument is rotated or moved around a superior articular process, the position sensor updates coordinates in real time and reduces an error.

The AI segmentation model unit 4 synchronizes position information between 2-D AP and lateral images and a 3-D image based on the data of the position sensor. Accordingly, a surgical operator can confirm an accurate position of instrument in the 2-D AP/lateral images, and can also check information on a position where the instrument is placed more intuitively because the corresponding position is displayed in the 3-D image in real time.

After step S34 is performed, a surgical process of the instrument is monitored (S36). In the monitoring process, the AI segmentation model unit 4 can derive the highlighting and warning of an important structure in order for a surgical operator to pay attention in real time by visually highlighting an important anatomical structure (e.g., a nerve or a blood vessel) and a danger section in the 3-D image. The warning of an important structure and the warning unit enable a surgical operator to visually recognize a danger section in real time based on the data of the position sensor, and prevent a danger in a surgical process, which is attributable to a surgical operator's carelessness or mistake, by providing a warning signal when the instrument approaches a specific structure or deviates a surgery path, thereby improving surgery safety.

A surgical operator determines whether an image of the C-arm equipment is matched with a scout image by overlaying the images of the C-arm equipment with the scout images, when monitoring the surgical process (S38 and S40). In this case, the surgical operator performs corrections until the images of the C-arm equipment are matched with the scout images when the images of the C-arm equipment are not matched with the scout images, and simulates a movement of the instrument when the images of the C-arm equipment are matched with the scout images (S42).

Finally, the prediction data of a moving path of the instrument are stored and analyzed (S44). In this case, the data record and analysis unit 12 stores and analyzes the data of the position sensor and the pressure sensor and information collected from the AP and lateral images generated by the AI segmentation model, which have been generated in the surgical process, after the surgical operation is performed. The collected data may be used to predict a change which may occur in a future surgical process and to optimize a system. Furthermore, the data that are recorded after the surgery are used for AI to perform learning so that Al can predict a path more accurately in a subsequent surgery. Accordingly, AI becomes increasingly sophisticated, thus contributing to the improvement of a surgical operator's skill.

It may be evident to a person having ordinary knowledge in the art to which the present disclosure pertains that the present disclosure described above is not limited by the aforementioned embodiments and the accompanying drawings and may be substituted, modified, and changed in various ways without departing from the technical spirit of the present disclosure.

### DESCRIPTION OF REFERENCE NUMERALS

2: scout image collection unit 4: AI segmentation model
6: sensor data and AP/lateral image synchronization module
8: instrument moving tracking module
10: instrument moving prediction simulation module
12: data record and analysis module
22: deep learning module
24: 3-D image generation module

What is claimed is:

1. A real-time surgery support computer system using a sensor-based artificial intelligence (AI) segmentation model, the system comprising:
a scout image collection unit configured to collect an anteroposterior (AP) image and lateral image of an anatomical structure of a spine by using C-arm equipment in order to perform supervised learning;
an AI segmentation model unit configured to generate a two-dimensional (2-D) image by learning the AP and lateral images collected by the scout image collection unit and then generate a lateral image from an AP image;
a sensor data and AP/lateral image synchronization module configured to synchronize AP/lateral images generated through the AI segmentation model unit and data of a position sensor and pressure sensor mounted on surgical instrument;
an instrument moving tracking module configured to visualize the data of the position sensor and pressure sensor synchronized with the AP/lateral images by tracking the data of the position sensor and pressure sensor; and
an instrument moving prediction simulation module configured to predict and simulate a movement of the surgical instrument that moves in the instrument moving tracking module in real time.

2. The real-time surgery support computer system of claim 1, further comprising a data record and analysis module configured to store and analyze data generated by the instrument moving prediction simulation module in order to use the data in learning so that a moving path of the surgical instrument is able to be predicted more accurately after a surgery.

3. The real-time surgery support computer system of claim 1, wherein the AI segmentation model unit comprises:
a deep learning module configured to generate the 2-D image, and
a three-dimensional (3-D) image generation module configured to generate the lateral image from the AP image by applying a mathematical formula model, a 3-D transform model, and a classification/minimum dimension model.

4. The real-time surgery support computer system of claim 2, wherein the deep learning module combines and trains a CNN model and an LSTM model.

5. The real-time surgery support computer system of claim 1, wherein the AI segmentation model unit further comprises a warning unit configured to predict a movement of the surgical instrument in the AP and lateral images and to generate a warning signal to a surgical operator when the surgical instrument deviates a safe region.

6. The real-time surgery support computer system of claim 1, wherein the surgical instrument comprises a trocar comprising:
a needle comprising a groove formed in a predetermined part and a long and thin pole having a pointed needle tip at a front end thereof;
a handle into which the needle is inserted and configured to apply a force so that directionality, straightness, and steerability are facilitated based on a percutaneous incident angle of a spinal facet joint;
a position sensor installed on one side of the handle and configured to generate coordinate data; and
a pressure sensor installed on the other side of the handle and configured to generate a force pattern.

7. The real-time surgery support computer system of claim 6, further comprising a mapping module configured to receive the coordinate data and data of the force pattern generated by the position sensor and pressure sensor of the trocar and to map the coordinate data and data of the force pattern to the AP and lateral images.

8. The real-time surgery support computer system of claim 7, wherein the mapping module comprises:
a computer main body configured to match the AP and lateral images and to perform a computing task;
a monitor configured to display AP and lateral images mapped in the computer main body; and
a keyboard configured to perform an operation of stopping a position of the surgical instrument in order to designate coordinate data when the surgical instrument with which the AP and lateral images of the AI segmentation model unit are overlaid is moved.

9. The real-time surgery support computer system of claim 1, wherein the surgical instrument comprises:
a needle comprising a groove formed in a predetermined part and a long and thin pole having a pointed needle tip at a front end thereof;
a handle into which the needle is inserted and configured to apply a force so that directionality, straightness, and steerability are facilitated based on a percutaneous incident angle of a spinal facet joint;
a position sensor installed on one side of the handle and configured to generate coordinate data; and
a pressure sensor installed on the other side of the handle and configured to generate a force pattern, and
wherein the handle comprises:
a cylinder comprising two space parts;
an adaptor coupled to one side of the cylinder and into which the needle is inserted; and
a hammer anvil installed on a cross section of the cylinder and configured to apply hammering in intervertebral foramen.

10. The real-time surgery support computer system of claim 9, wherein:
the pressure sensor is installed in the space part of the cylinder on one side thereof,
the space part of the cylinder on the other side thereof is formed as a pressure transfer space, and
a shock buffer is installed in the pressure transfer space and generates a signal by reducing strength of striking pressure of the hammering that is transferred to the pressure sensor.

11. The real-time surgery support computer system of claim 9, wherein:
a sensor connector is mounted on the space part of the cylinder on the other side thereof, and
a circuit board and a battery are embedded in the sensor connector.

12. The real-time surgery support computer system of claim 9, wherein:
a cover is installed at an end of the cylinder on one side thereof, and
a position sensor, a power switch, LED lamps for indicating a charging state, and an antenna for communicating with the AI segmentation model unit are mounted on the cover.

13. The real-time surgery support computer system of claim 9, wherein a predetermined interval is formed between the adaptor and the pressure transfer space so that pressure is collected in the pressure transfer space and transferred to the needle when the striking for the hammering is applied to the cylinder in a state in which the adaptor and the pressure sensor have not come into direct contact with each other.

14. The real-time surgery support computer system of claim 9, wherein the position sensor comprises a load cell and a gyroscope in order to generate position coordinates.

15. The real-time surgery support computer system of claim 9, wherein:
the position sensor and the pressure sensor that are installed in the handle are connected to a mapping module and transmit a position signal and a pressure signal, and
the mapping module comprises:
a computer main body configured to match the AP and lateral images and to perform a computing task,
a monitor configured to display AP and lateral images mapped in the computer main body, and
a keyboard configured to perform an on/off operation for designating a position of the surgical instrument in order to designate reference coordinate data when the surgical instrument with which the AP and lateral images of the AI segmentation model unit are overlaid is moved.

16. The real-time surgery support computer system of claim 1, wherein the AI segmentation model unit comprises one or more learning model selected, among a transformer model, 3-D convolutional neural networks (3-D CNNs), a recurrent convolutional neural network (RCNN), generative adversarial networks (GANs), attention mechanisms, autoencoders, and reinforced learning.

17. A real-time surgical support method using a sensor-based artificial intelligence (AI) segmentation model, the method comprising:
a first step of collecting scout anteroposterior (AP) and lateral images of a spine from C-arm equipment;
a second step of classifying and labelling each tissue region of an anatomical structure by pre-processing the scout AP and lateral images;
a third step of obtaining AP and lateral images by learning the labelled scout AP and lateral images in an AI segmentation model;
a fourth step of obtaining position data of instrument from a scout AP image that is initially collected, mapping the position data of the instrument to a scout lateral image, and visualizing the position data of the instrument;
a fifth step of designating a reference point of the position data of the instrument in the scout lateral image;
a sixth step of proposing an optimal moving path of the instrument to a surgical operator through a chatbot function of the AI segmentation model after performing the fifth step;
a seventh step of determining whether the instrument has deviates its moving path after performing the sixth step, generating warning when the instrument deviates a safe region, and automatically correcting the moving path, if necessary;
an eighth step of obtaining, by an instrument movement simulation module, force pattern data from a pressure sensor while continuously simulating the moving path of the instrument until a trocar reaches a target position, after performing the seventh step; and
a ninth step of determining, by a surgical operator upon monitoring, whether the images of the C-arm equipment are matched with the scout images by overlaying the images of the C-arm equipment, performing corrections until the images of the C-arm equipment are matched with the scout images when the images of the C-arm equipment are not matched with the scout images, and simulating a movement of the instrument when the images of the C-arm equipment are matched with the scout images.

18. The real-time surgical support method of claim 17, wherein the learning of the AI segmentation model comprises:
a first process of learning the AP/lateral images of the anatomical structure through a CNN algorithm and an LSTM algorithm that are included in a deep learning module;
a second process of learning an AP image and lateral image generated by a three-dimensional (3-D) image generation module and generating a lateral image (or an AP image) from the AP image (or the lateral image) by using a mathematical formula algorithm and a 3-D transform algorithm;
a third process of integrating the AP image and the lateral image so that a surgical operator is able to view the AP image and the lateral image on one screen; and
a fourth process of completing a scout segmentation image by integrating the AP image and the lateral image.

19. The real-time surgical support method of claim 17, wherein the designating of the reference point of the position data comprises designating a hammer point, a facet line, and a swing position which are classification areas of the anatomical structure.

20. The real-time surgical support method of claim 17, wherein the pressure sensor detects pressure that is applied to a specific tissue, and provides feedback for excessive pressure to the surgical operator in real time when the excessive pressure occurs.

21. The real-time surgical support method of claim 17, wherein in order to accurately track a movement of the instrument based on data of a position sensor and the pressure sensor, the AI segmentation model understands and learns a process by labelling a movement of the instrument in a coded language.

22. The real-time surgical support method of claim 17, wherein when a movement of the trocar deviates in an unexpected direction or pressure of the trocar is suddenly changed, the AI segmentation model immediately detects the movement or the pressure and indicates warning in order to prevent an insertion of the trocar in a wrong direction or excessive pressure of the trocar.

23. The real-time surgical support method of claim 17, further comprising a tenth step of storing and analyzing data of a position sensor and the pressure sensor generated in a surgical process and information collected from an AI segmentation model and the AP and lateral images in order to store and analyze data of predicting a moving path of the instrument.

24. A computer-readable recording medium on which a computer program for performing the real-time surgical support method using a sensor-based AI segmentation model according to claim 17 has been recorded.
